(19) United States
(12) Patent Application Publication
(10) Pub. No.: US 2024/0069311 A1
(43) Pub. Date: Feb. 29, 2024

(54) METHOD OF MANUFACTURING LENS UNIT, LENS UNIT, IMAGE PICKUP APPARATUS, AND ENDOSCOPE

(71) Applicant: OLYMPUS CORPORATION, Tokyo (JP)

(72) Inventor: Jumpei YONEYAMA, Kamiina-gun (JP)

(73) Assignee: OLYMPUS CORPORATION, Tokyo (JP)

(21) Appl. No.: 18/387,251

(22) Filed: Nov. 6, 2023

Related U.S. Application Data
(63) Continuation of application No. PCT/JP2021/027905, filed on Jul. 28, 2021.

(51) Int. Cl. G02B 13/00; A61B 1/00

(52) U.S. Cl. CPC G02B 13/0085; A61B 1/00096; A61B 1/0011

(57) ABSTRACT

A method of manufacturing a lens unit includes: producing a stacked wafer including a plurality of optical wafers that include at least one optical wafer in which a light shielding layer is disposed and having a first principal surface and a second principal surface; forming grooves in a grid pattern on the first principal surface or the second principal surface of the stacked wafer using a dicing blade, in which the grooves have a depth at which the light shielding layer is cut; and dividing the stacked wafer into a plurality of lens units by performing stealth dicing using a filamentation laser along the grooves.

### CROSS REFERENCE TO RELATED APPLICATION

This application is a continuation application of PCT/JP2021/027905 filed on Jul. 28, 2021, the entire contents of which are incorporated herein by this reference.

### BACKGROUND OF THE INVENTION

### 1. Field of the Invention

The present invention relates to a method of manufacturing a lens unit including a step on a side surface, the lens unit including the step on the side surface, an image pickup apparatus including the lens unit including the step on the side surface, and an endoscope having the image pickup apparatus including the lens unit including the step on the side surface.

### 2. Description of the Related Art

It is important to reduce a diameter of a lens unit of an image pickup apparatus disposed in a distal end portion of an endoscope for alleviating invasiveness.

International Publication No. 2017/203592 discloses a lens unit that is a wafer-level stacked body that allows efficient manufacturing of the lens unit with a small-diameter. The wafer-level stacked body is manufactured by cutting a stacked wafer in which a plurality of optical wafers, each including a plurality of lens devices, are stacked.

Japanese Patent Application Laid-Open Publication No. 2009-072829 discloses a cutting method using a filamentation laser capable of performing a high-speed treatment without causing chipping on a glass.

### SUMMARY OF THE INVENTION

A method of manufacturing a lens unit of an embodiment includes: producing a stacked wafer including a plurality of optical wafers that include at least one optical wafer in which a light shielding layer constituting an aperture is disposed on a glass wafer, and having a first principal surface and a second principal surface on a side opposite to the first principal surface; forming grooves in a grid pattern on the first principal surface or the second principal surface of the stacked wafer using a dicing blade, in which the grooves have a depth at which the light shielding layer is cut; and dividing the stacked wafer into a plurality of lens units by performing stealth dicing using a filamentation laser along the grooves.

A lens unit of an embodiment includes a plurality of optical devices including at least one hybrid lens device including a glass substrate, a light shielding layer constituting an aperture, and a resin lens, and four side surfaces of the lens unit each include: a first region in which a side surface of the light shielding layer is exposed and which has a striation inclined relative to an optical axis direction; and a second region which is positioned further from an optical axis than the first region and is free from the striation.

An image pickup apparatus of an embodiment includes a lens unit and an image pickup unit, in which the lens unit includes a plurality of optical devices including at least one hybrid lens device including a glass substrate, a light shielding layer constituting an aperture, and a resin lens, and four side surfaces of the lens unit each include: a first region in which a side surface of the light shielding layer is exposed and which has a striation inclined relative to an optical axis direction; and a second region which is positioned further from an optical axis than the first region and is free from the striation.

An endoscope of an embodiment includes an image pickup apparatus that includes a lens unit and an image pickup unit, in which the lens unit includes a plurality of optical devices including at least one hybrid lens device including a glass substrate, a light shielding layer constituting an aperture, and a resin lens, and four side surfaces of the lens unit each include: a first region in which a side surface of the light shielding layer is exposed and which has a striation inclined relative to an optical axis direction; and a second region which is positioned further from an optical axis than the first region and is free from the striation.

### BRIEF DESCRIPTION OF THE DRAWINGS

FIG. 1 is a perspective view of an image pickup apparatus of a first embodiment;

FIG. 2 is a cross-sectional view taken along line II-II of FIG. 1;

FIG. 3 is a flowchart of a manufacturing method of the image pickup apparatus of the first embodiment;

FIG. 4 is an exploded perspective view for explaining the manufacturing method of the image pickup apparatus of the first embodiment;

FIG. 5 is a cross-sectional view for explaining the manufacturing method of the image pickup apparatus of the first embodiment;

FIG. 6 is a cross-sectional view for explaining the manufacturing method of the image pickup apparatus of the first embodiment;

FIG. 7 is a cross-sectional view of an image pickup apparatus of a modification 1 of the first embodiment;

FIG. 8 is a cross-sectional view of an image pickup apparatus of a modification 2 of the first embodiment; and

FIG. 9 is a perspective view of an endoscope of a second embodiment.

### DETAILED DESCRIPTION OF THE PREFERRED EMBODIMENTS

### First Embodiment

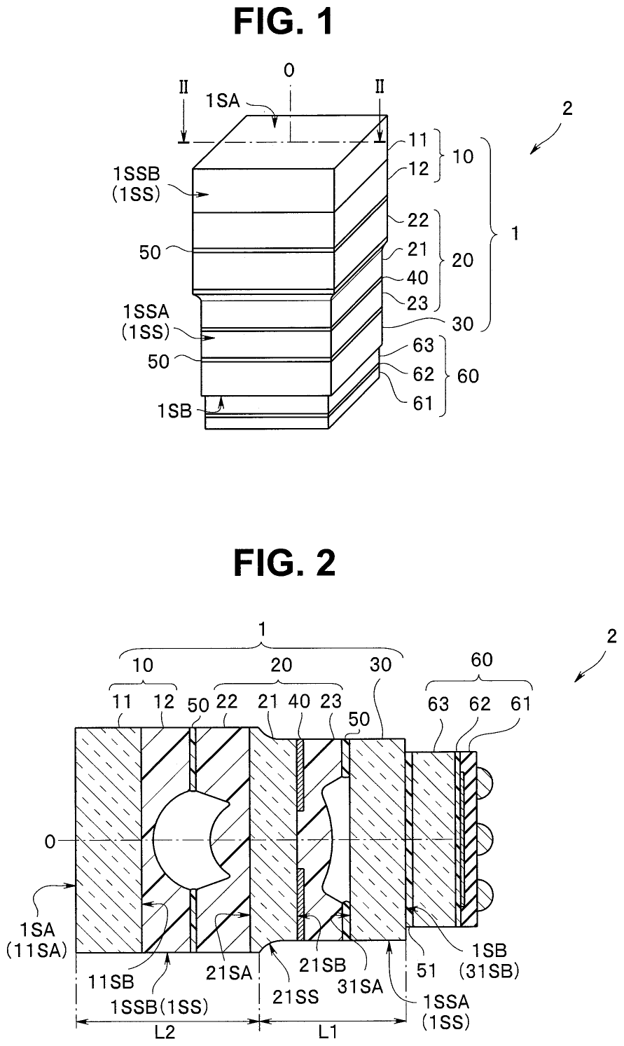

An image pickup apparatus 2 of an embodiment shown in FIG. 1 and FIG. 2 includes a lens unit 1 and an image pickup unit 60 of the embodiment. A reference sign O indicates an optical axis of the lens unit 1. The image pickup unit 60 receives light of a subject image condensed by the lens unit 1 to convert the subject image into an image pickup signal.

Note that in the following description, the drawings based on the embodiments are schematic illustrations. The relation between the thickness and the width of each portion, the ratio in thickness and the relative angle of each portion, and the like differ from the actual components. There are also some portions with different dimensional relations and ratios among the drawings. Illustration of part of the constituent elements will be omitted.

The lens unit 1 includes a first optical device 10 including an incident surface 1SA, a second optical device 20, and a third optical device 30 including an emission surface 1SB. The first optical device 10, the second optical device 20, and the third optical device 30 are stacked in this order.

The first optical device 10 includes, as a base body, a first glass substrate 11 having a first principal surface 11SA as the incident surface 1SA and a second principal surface 11SB on a side opposite to the first principal surface 11SA. The first optical device 10 is a hybrid lens device including a resin lens 12 that is a concave lens on the second principal surface 11SB.

The second optical device 20 includes, as a base body, a second glass substrate 21 having a third principal surface 21SA and a fourth principal surface 21SB on a side opposite to the third principal surface 21SA. The third principal surface 21SA is disposed facing the second principal surface 11SB. The second optical device 20 is a hybrid lens device including a resin lens 22 that is a convex lens on the third principal surface 21SA and a resin lens 23 that is a convex lens on the fourth principal surface 21SB. A light shielding layer 40 that is made of metal with chromium or titanium as a main component and forms an aperture is disposed on the fourth principal surface 21SB.

The third optical device 30 is a third glass substrate 31 having a fifth principal surface 31SA and a sixth principal surface 31SB as the emission surface 1SB on a side opposite to the fifth principal surface 31SA. The fifth principal surface 31SA is disposed facing the fourth principal surface 21SB.

The first glass substrate 11, the second glass substrate 21, and the third glass substrate 31 are made of, for example, borosilicate glass, quartz glass, or sapphire glass.

The first optical device 10 and the second optical device 20, and the second optical device 20 and the third optical device 30 are respectively adhesively bonded by means of an adhesive layer 50 made of resin.

Note that the configuration of the lens unit of the present invention is not limited to the configuration of the lens unit 1 of the present embodiment, and is determined in accordance with the specification. For example, the lens unit may include a spacer element that defines a distance between the lenses and a plurality of light shielding layers in addition to the lens device.

The image pickup unit 60 is adhesively bonded to the sixth principal surface 31SB (emission surface 1SB) of the third optical device 30 by means of an adhesive layer 51. In the image pickup unit 60, a cover glass 63 is adhesively bonded to an image pickup device 61 by means of an adhesive layer 62. The lens unit 1 forms a subject image on the image pickup device 61. The image pickup device 61 is a CMOS (complementary metal oxide semiconductor) light receiving element or a CCD (charge coupled device).

Four side surfaces 1SS of the lens unit 1 each include a first region 1SSA in which a side surface of the light shielding layer 40 is exposed and which has a striation inclined relative to an optical axis direction, and a second region 1SSB which is positioned further from the optical axis O than the first region 1SSA and is free from the striation.

The striation is a characteristic of a cut surface obtained by cutting the first region 1SSA by a first method using a dicing blade. Meanwhile, the second region is a cut surface obtained by cutting the second region by a second method without using the dicing blade.

Since the light shielding layer 40 is cut by the first method using the dicing blade, the side surface is exposed in the first region 1SSA.

As will be described later, a cutting margin formed by the second method is smaller than a cutting margin formed by the first method. Therefore, the second region 1SSB is positioned further from the optical axis O than the first region 1SSA. In other words, there is a step at a boundary between the first region 1SSA and the second region 1SSB on the side surface 1SS (side surface 21SS of the second glass substrate 21) of the lens unit 1.

Note that a length L1 of the first region 1SSA in a direction parallel to the optical axis is shorter than a length L2 of the second region 1SSB. In other words, the length L2 of the side surface of the lens unit 1 formed by stealth dicing is longer than a depth L1 of a groove formed using the dicing blade.

In the lens unit 1, the second method is stealth dicing using a filamentation laser. The light shielding layer 40 that cannot be cut using a laser is cut by the first method using the dicing blade. The incident surface 1SA to be cut last, in which chipping is most likely to occur, is cut by the second method that prevents chipping on the glass substrate, and thus, the lens unit 1 is easy to manufacture and is highly reliable.

<Manufacturing Method>

The lens unit 1 is a wafer-level optical unit that is manufactured by cutting a stacked wafer 1W in which a plurality of optical wafers, each including a plurality of optical devices disposed in a matrix, are stacked.

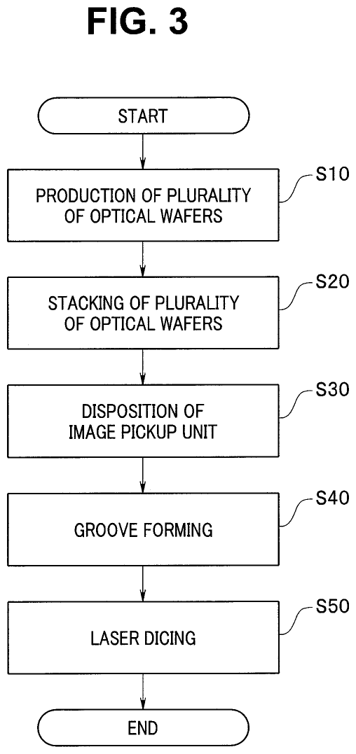

Hereinafter, a manufacturing method of the image pickup apparatus 2 by cutting a stacked wafer 2W in which the plurality of image pickup units 60 are disposed on the stacked wafer 1W will be described as an example following a flowchart of FIG. 3.

<Step S10> Production of a Plurality of Optical Wafers

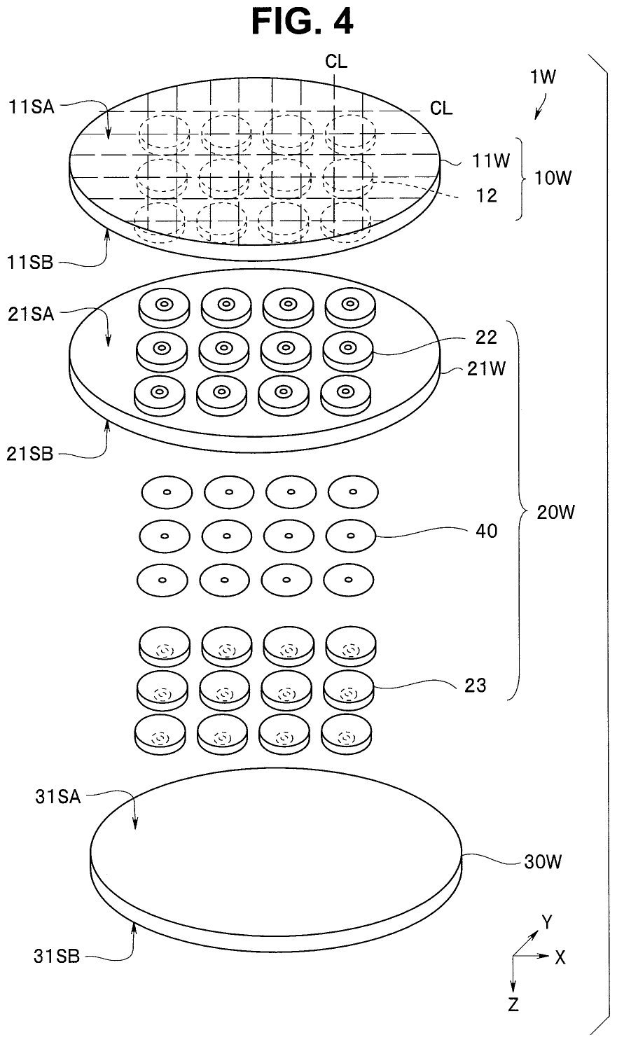

As shown in FIG. 4, the plurality of resin lenses 12 are disposed on the second principal surface 11SB of a glass wafer 11W, so that an optical wafer 10W is produced. A reference sign CL indicates a plurality of cut lines in a grid pattern. It is preferable that energy curable resin should be used for the resin lens 12.

Cross-linking reaction or polymerization reaction of the energy curable resin proceeds by reception of energy such as heat, ultraviolet light, and electron beam from outside. For example, the energy curable resin includes transparent ultraviolet curing silicone resin, epoxy resin, or acrylic resin. Note that “transparent” means that a material has less light absorption and less scattering in such a degree that the material can endure in use in a use wavelength range.

The resin lens 12 is produced using a mold method in which uncured resin, which is thus liquid or gel, is disposed on the glass wafer 11W and ultraviolet light is irradiated to cure the resin in a state of being pressed by a mold having a recessed portion with a predetermined inner surface shape. Note that silane coupling treatment or the like is preferably performed on the glass wafer before the resin is disposed to improve an interface adhesive strength between the glass and the resin.

Since an inner surface shape of the mold is transferred to an outer surface shape of the resin lens manufactured using the mold method, it is possible to easily produce the configuration having an outer edge portion which also functions as a spacer and an aspherical lens.

An optical wafer 20W is produced in a similar manner as the optical wafer 10W. In the optical wafer 20W, the light shielding layer 40 is disposed on the fourth principal surface 21SB of a glass wafer 21W before the resin lens 23 is disposed. For example, a metal layer disposed on the fourth principal surface 21SB is patterned using a sputtering method, so that the plurality of light shielding layers 40 are produced. The light shielding layer 40 includes chromium or titanium as a main component. The “main component” means accounting for 90% or more by weight. A thickness of the light shielding layer 40 is, for example, 0.2 μm to 2 μm for securing the light shielding property.

<Step S20> Wafer Stacking

As shown in FIG. 4, the optical wafer 10W, the optical wafer 20W, and the optical wafer 30W are stacked. Though not shown, the adhesive layer 50 is disposed on each of the resin lens 12 of the optical wafer 10W, and the resin lens 22 and the resin lens 23 of the optical wafer 20W using a transfer method. The adhesive layer 50 may be disposed using an inkjet method, for example. The adhesive layer 50 is, for example, thermosetting epoxy resin. The optical device wafers (optical wafers) 10W to 30W are stacked and adhesively bonded together, so that the stacked wafer 1W is produced. The stacked wafer 1W includes the incident surface 1SA and the emission surface 1SB on a side opposite to the incident surface 1SA.

<Step S30> Disposition of Image Pickup Unit

The plurality of image pickup units 60 are adhesively bonded to the emission surface 1SB (sixth principal surface 31SB) of the stacked wafer 1W using the adhesive layer 51, so that the stacked wafer 2W is produced.

The image pickup unit 60 is manufactured by cutting an image pickup wafer in which a glass wafer is adhesively bonded, using a transparent adhesive, to an image pickup device wafer including a plurality of light receiving circuits. Note that the stacked wafer 2W may be produced by adhesively bonding the image pickup wafer to the stacked wafer 1W.

<Step S40> Groove Forming

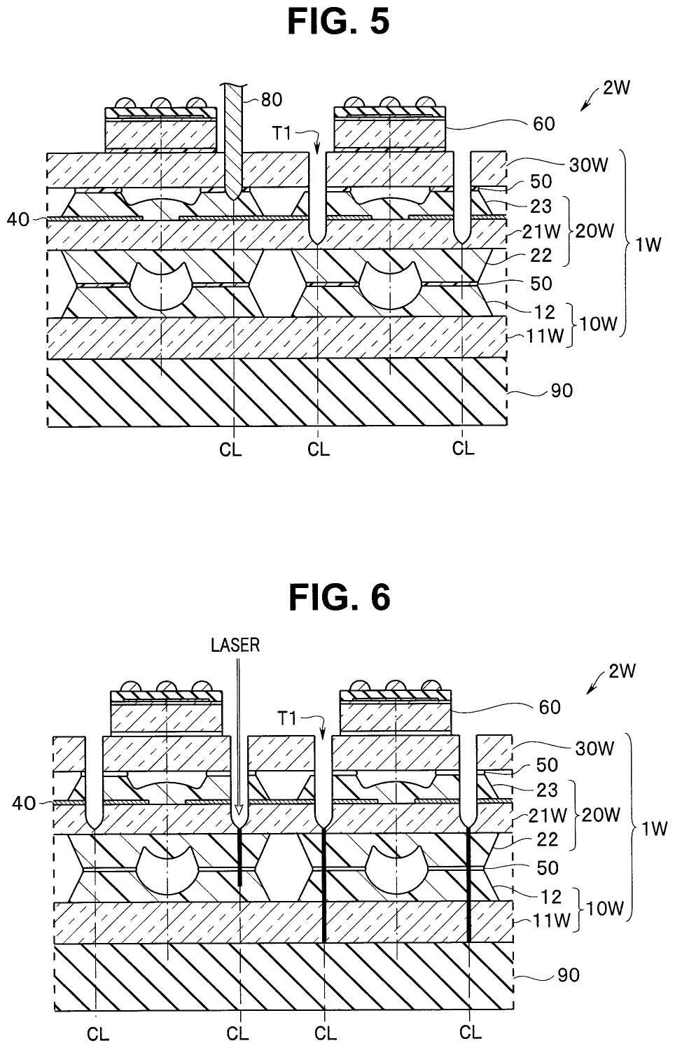

As shown in FIG. 5, in the stacked wafer 1W, the incident surface 1SA (first principal surface 11SA) of the optical wafer 10W is attached to a fixation member such as a dicing tape 90. Then, a dicing blade 80 is used to form, on the stacked wafer 2W, grooves T1 having a depth at which the light shielding layer 40 is cut along the cut lines CL in a grid pattern.

<Step S50> Laser Dicing

As shown in FIG. 6, stealth dicing is performed along the grooves T1 (cut lines CL) using a filamentation laser, so that the stacked wafer 2W is divided into the plurality of lens units 1.

The filamentation is a distinctive phenomenon of a highly-intense femtosecond laser. With a dynamic non-linear optical effect in which light is propagated while converging and diverging in balance, condensed light is propagated as it is in a long distance, so that a linear plasm is generated. Therefore, in the stacked wafer 2W subjected to stealth dicing with filamentation laser scanning, a modified region is created in a scanning direction. The stacked wafer 2W in which the modified region is created is divided into the plurality of lens units 1 in the scanning direction, that is, along the grooves T1, with the stress externally applied. For the division, the stress applied to the stacked wafer 2W may be mechanically applied or may be a stress generated by thermal treatment.

A width (cutting margin) of the groove T1 formed using the dicing blade 80 is 50 μm to 200 μm, while the cutting margin formed using a filamentation laser is only 1 μm to 3 μm. In the second region 1SSB divided using the filamentation laser, principally, machining marks parallel to the optical axis direction are formed. However, the machining marks are often not clearly observed due to the extreme fineness or the like.

The image pickup apparatus 2 is manufactured by a wafer-level method and thus, has a small-diameter and is easy to manufacture. Further, the optical wafer 10W, in which chipping is particularly likely to occur and which is attached to the dicing tape 90 in cutting the stacked wafer 1W, is divided by stealth dicing using a filamentation laser. Therefore, the lens unit 1 and the image pickup apparatus 2 are easy to manufacture and are highly reliable because the glass substrate is free from chipping.

Note that the image pickup apparatus 2 may be produced by disposing the image pickup unit 60 on the lens unit 1 manufactured by cutting the stacked wafer 1W.

In cutting the stacked wafer 1W, it is also possible to form grooves on the incident surface 1SA using a dicing blade and divide the stacked wafer 1W along the grooves by stealth dicing. However, the length L2 of the second region 1SSB formed by stealth dicing is shorter than the depth of the grooves (length L1 of the first region) formed using the dicing blade. Therefore, the time period required for the groove formation is longer than when the grooves are formed on the emission surface. For this reason, the grooves are preferably formed on the emission surface. In other words, the length L2 of the second region 1SSB formed by stealth dicing is preferably longer than the depth of the grooves (length L1 of the first region) formed using the dicing blade.

### Modifications of First Embodiment

Lens units 1A, 1B and image pickup apparatuses 2A, 2B of modifications of the first embodiment are similar to and have the same effects as the effects of the lens unit 1 and the image pickup apparatus 2. Therefore, the constituent elements having the same functions will be assigned the same reference numerals, and the description will be omitted.

### Modification 1 of First Embodiment

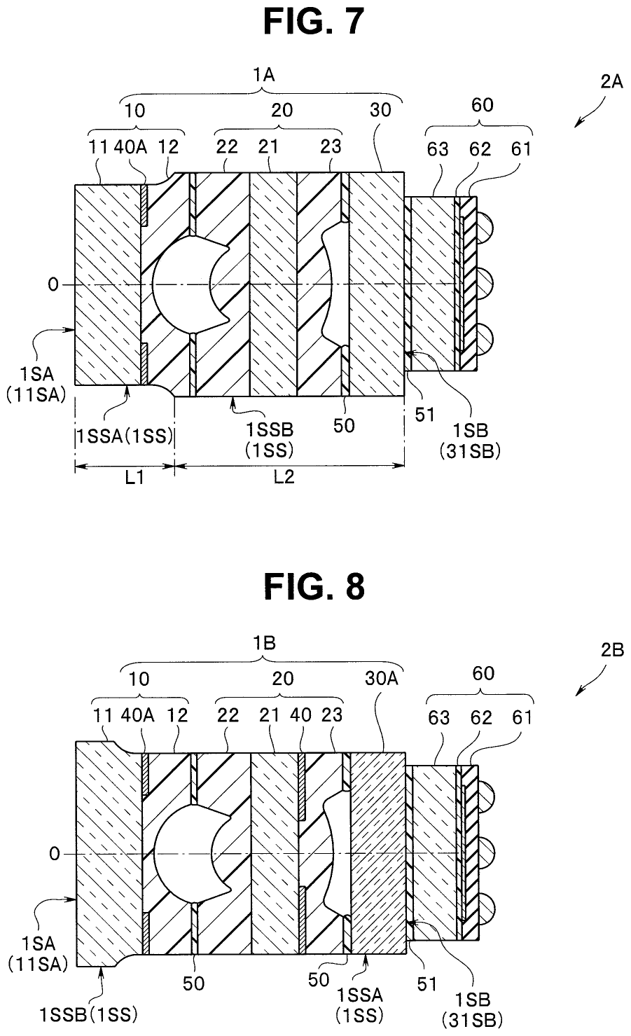

In the image pickup apparatus 2A (lens unit 1A) of the present modification shown in FIG. 7, a light shielding layer 40A is disposed on the first optical device 10. In other words, the light shielding layer 40A is disposed on the second principal surface 11SB of the first glass substrate 11.

Though not shown, in manufacturing the lens unit 1A, the emission surface 1SB of the stacked wafer 1W is fixed to a dicing tape. Grooves having a depth at which the light shielding layer 40A is cut are formed in a grid pattern on the incident surface 1SA. The stacked wafer 1W is divided into the lens units 1A by stealth dicing that involves irradiation of a filamentation laser along the grooves. Then, the image pickup unit 60 is disposed on the divided lens unit 1A, so that the image pickup apparatus 2A is produced.

In the lens unit 1A, the grooves are formed on the incident surface 1SA. The length L1 of the first region 1SSA of the side surface 1SS cut by blade dicing is shorter than the length L2 of the second region 1SSB of the side surface 1SS cut by stealth dicing. Therefore, the time period required for cutting the lens unit 1A is short.

### Modification 2 of First Embodiment

In the image pickup apparatus 2B (lens unit 1B) of the present modification shown in FIG. 8, the light shielding layers 40, 40A as an adhesive layer made of light shielding resin are disposed on the first optical device 10 and the second optical device 20. A third optical device 30A is a filter device that removes unnecessary infrared light (for example, light with a wavelength equal to or greater than 700 nm). The third optical device 30A cannot be cut using a laser.

Though not shown, in manufacturing the lens unit 1B, the incident surface 1SA of the stacked wafer 2W is fixed to the dicing tape 90. Grooves having a depth at which a filter wafer and the light shielding layers 40, 40A are cut are formed in a grid pattern on the emission surface 1SB. A bottom surface of the grooves is positioned within a first glass wafer.

In the lens unit 1B, side surfaces of the light shielding layers 40, 40A and a side surface of the third optical device 30A as the filter device are exposed in the first region 1SSA as wall surfaces of the grooves formed using the dicing blade.

The optical wafer 10W, in which chipping is particularly likely to occur and which is attached to the dicing tape 90, is divided by stealth dicing using a filamentation laser. Therefore, the lens unit 1B and the image pickup apparatus 2B are easy to manufacture and are highly reliable because the first glass substrate 11 is free from chipping.

### Second Embodiment

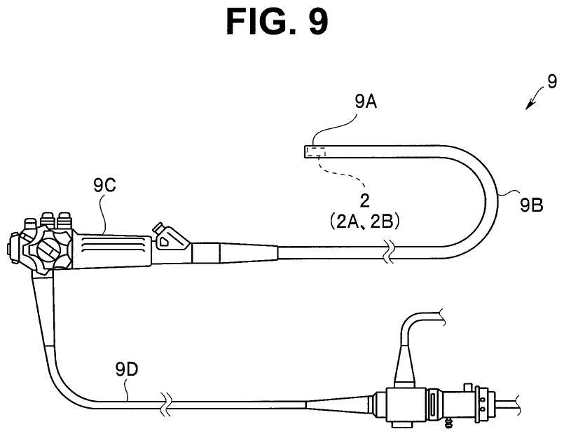

An endoscope 9 of the present embodiment shown in FIG. 9 includes a distal end portion 9A, an insertion portion 9B extending from the distal end portion 9A, an operation portion 9C disposed on a proximal end side of the insertion portion 9B, and a universal cord 9D extending from the operation portion 9C. The image pickup apparatus 2 (2A, 2B) including the lens unit 1 (1A, 1B) is disposed in the distal end portion 9A. An image pickup signal outputted from the image pickup apparatus 2 is transmitted to a processor (not shown) via a cable that passes through the universal cord 9D. A drive signal from the processor to the image pickup apparatus 2 is also transmitted via the cable that passes through the universal cord 9D.

The endoscope 9 may be a flexible endoscope with the insertion portion 9B that is flexible or a rigid endoscope with the insertion portion 9B that is rigid. The endoscope 9 may be for either medical use or industrial use.

The endoscope 9 includes the image pickup apparatus 2 (2A, 2B) including the lens unit 1 (1A, 1B), and thus, is easy to manufacture and is highly reliable.

The present invention is not limited to the aforementioned embodiments and the like, and various changes, combinations, and applications are available within the scope without departing from the gist of the invention.

What is claimed is:

1. A method of manufacturing a lens unit, comprising:
producing a stacked wafer including a plurality of optical wafers including at least one optical wafer in which a light shielding layer constituting an aperture is disposed on a glass wafer, the stacked wafer having a first principal surface and a second principal surface on a side opposite to the first principal surface;
forming grooves in a grid pattern on the first principal surface or the second principal surface of the stacked wafer using a dicing blade, the grooves having a depth at which the light shielding layer is cut; and
dividing the stacked wafer into a plurality of lens units by performing stealth dicing using a filamentation laser along the grooves.

2. The method of manufacturing a lens unit according to claim 1, wherein
the stacked wafer includes a plurality of light shielding layers, and
all the plurality of light shielding layers included in the stacked wafer are cut by forming the grooves.

3. The method of manufacturing a lens unit according to claim 1, wherein
the stacked wafer includes a filter wafer, and
the filter wafer is cut by forming the grooves.

4. The method of manufacturing a lens unit according to claim 2, wherein
the stacked wafer includes an adhesive layer made of light shielding resin configured to adhesively bond the plurality of optical wafers together, and
the adhesive layer is cut by forming the grooves.

5. The method of manufacturing a lens unit according to claim 2, wherein a bottom surface of the grooves is irradiated with the filamentation laser.

6. The method of manufacturing a lens unit according to claim 5, wherein a length of a side surface of the lens unit formed by stealth dicing is longer than a depth of the grooves formed using the dicing blade.

7. A lens unit comprising a plurality of optical devices including at least one hybrid lens device including a glass substrate, a light shielding layer constituting an aperture, and a resin lens, wherein
four side surfaces of the lens unit each include:
a first region in which a side surface of the light shielding layer is exposed, the first region having a striation inclined relative to an optical axis direction; and
a second region positioned further from an optical axis than the first region, the second region being free from the striation.

8. The lens unit according to claim 7, wherein
the plurality of optical devices include a filter device, and
the first region includes a side surface of the filter device.

9. An image pickup apparatus comprising:
a lens unit, and
an image pickup unit, wherein
the lens unit includes a plurality of optical devices including at least one hybrid lens device including a glass substrate, a light shielding layer constituting an aperture, and a resin lens, wherein
four side surfaces of the lens unit each include:
a first region in which a side surface of the light shielding layer is exposed, the first region having a striation inclined relative to an optical axis direction; and
a second region positioned further from an optical axis than the first region, the second region being free from the striation.

10. An endoscope comprising an image pickup apparatus including a lens unit and an image pickup unit, wherein
the lens unit includes a plurality of optical devices including at least one hybrid lens device including a glass substrate, a light shielding layer constituting an aperture, and a resin lens, wherein
four side surfaces of the lens unit each include:
a first region in which a side surface of the light shielding layer is exposed, the first region having a striation inclined relative to an optical axis direction; and
a second region positioned further from an optical axis than the first region, the second region being free from the striation.
